(19) United States
(12) Patent Application Publication
(10) Pub. No.: US 2012/0209115 A1
(43) Pub. Date: Aug. 16, 2012

(54) ULTRASONIC DIAGNOSTIC DEVICE, METHOD FOR GENERATING IMAGE FOR EVALUATING DISORDER OF PART TO BE DIAGNOSED OF OBJECT, AND PROGRAM FOR GENERATING IMAGE FOR EVALUATING DISORDER OF PART TO BE DIAGNOSED OF OBJECT

(75) Inventor: Akiko Tonomura, Tokyo (JP)

(73) Assignee: HITACHI MEDICAL CORPORATION, Tokyo (JP)

(21) Appl. No.: 13/500,991

(22) PCT Filed: Oct. 15, 2010
(86) PCT No.: PCT/JP2010/068136

§ 371 Date: Apr. 9, 2012

(30) Foreign Application Priority Data
Oct. 30, 2009 (JP) 2009-249714

(51) Int. Cl. A61B 8/00

(52) U.S. Cl. 600/438

(57) ABSTRACT

An ultrasonic diagnostic device is provided with: an ultrasonic probe which transmits and receives ultrasonic waves to and from an object; a reception processing unit which receives a reflection echo signal measured by the ultrasonic probe and generates RF signal frame data relating to a cross-sectional plane of a part to be diagnosed of the object; a displacement measurement unit which measures the displacements of tissue at a plurality of measurement points of the cross-sectional plane and generates displacement frame data, an elasticity information calculation unit which calculates elasticity information indicating the hardness or softness of the tissue at the plurality of measurement points and generates elasticity frame data; a unit for generating image which generates histograms of the displacements and/or the elasticity information of the tissue at the plurality of measurements points at different times; and an image display which displays the histograms generated at the different times.

### TECHNICAL FIELD

The present invention relates to an ultrasonic diagnostic device, a method for generating an image for evaluating a disorder of a part to be diagnosed of an object to be examined, and a program for generating an image for evaluating a disorder of a part to be diagnosed of an object and, more particularly, to a technique for improving the quantitativity of an image for evaluation used to evaluate the degree of a disorder of a part to be diagnosed of an object.

### BACKGROUND ART

An ultrasonic diagnostic device is intended to transmit ultrasonic waves to the interior of an object to be examined by an ultrasonic probe including a plurality of ultrasonic transducers, receive reflection echo signals corresponding to the structure of a living organism tissue from the interior of the object, and generate a tomographic image such as a B-mode image on the basis of the reflection echo signals to display the tomographic image for diagnosis.

In recent years, the process of measuring ultrasonic reception signals (RF signals) while manually or mechanically pressing an object with an ultrasonic probe and generating an elasticity image indicating the hardness or softness of a tissue at a cross-sectional plane has been performed, as disclosed in Patent Literature 1. More specifically, the process of obtaining displacements at parts of a tissue caused by pressing on the basis of a pair of pieces of RF signal frame data having different states of pressing against the tissue, calculating frame data of elasticity information such as a strain amount or an elastic modulus on the basis of frame data of the obtained displacements, and generating and displaying an elasticity image on the basis of the elasticity frame data has been performed.

An elasticity image is expected to be used not only in diagnosis of a mass lesion such as cancer but also in diagnosis of a diffuse disorder. More specifically, if local hardened tissues such as a tuber are scattered in a surrounding soft tissue in the case of a diffuse disorder, a patchy pattern of the hardened tissues is reflected in an elasticity image. For example, if a disorder progresses, e.g., from hepatitis to liver cirrhosis to cause fibrotic progression, tubers spread into a liver parenchyma, and a patchy pattern of hardened tissues in an elasticity image becomes complicated. A tester observes the elasticity image and makes evaluations of the degree of a disorder of a part to be diagnosed, the degree of progression of the disorder, effects of treatment of the disorder, and the like (hereinafter collectively referred to as disorder evaluation as appropriate) on the basis of the state of the patchy pattern of the hardened tissues in the elasticity image.

However, if disorder evaluation is visually performed by a tester, results of the disorder evaluation vary among testers. The process of allowing objective disorder evaluation is thus desired.

In this regard, the process of displaying as a histogram a distribution of elasticity information in a region of interest set in an elasticity image is known, as disclosed in, e.g., Patent Literature 2. The process allows provision of new quantitative information, a distribution of elasticity information for a tissue of an object, in addition to provision of elasticity information at a cross-sectional plane of the tissue as an elasticity image.

### CITATION LIST

### Patent Literature

Patent Literature 1: Japanese Patent Laid-Open No. 5-317313
Patent Literature 2; International Publication No. WO 2007/046272

### SUMMARY OF INVENTION

### Technical Problem

However, there is a need for improving the technique in Patent Literature 2 so as to allow a tester to more quantitatively perform disorder evaluation of a part to be diagnosed of an object.

More specifically, a tester may be unable to easily perform quantitative disorder evaluation of a part to be diagnosed of an object by just displaying as a histogram a distribution of elasticity information while picking up an elasticity image, as in Patent Literature 2. For example, it is not easy to evaluate, by just taking one look at a histogram for a current object, how far a disorder of a part to be diagnosed has progressed from the previous test or how much treatment has been effective. Although just one look at a histogram may allow rough evaluation of the degree of a disorder of a current part to be diagnosed, more quantitative evaluation is hard to implement with just one look.

Under the circumstances, a subject of the present invention is to provide an image for evaluation for more quantitatively perform disorder evaluation of a part to be diagnosed of an object.

### Solution to Problem

In order to achieve the above subjects, an ultrasonic diagnostic device according to the present invention includes: an ultrasonic probe which transmits/receives ultrasonic waves to/from an object; reception processing unit for receiving a reflection echo signal measured by the ultrasonic probe and generating RF signal frame data at a cross-sectional plane of a part to be diagnosed of the object; displacement measurement unit for, on the basis of one pair of pieces of RF signal frame data having different states of pressing against a tissue at the cross-sectional plane, measuring displacements of the tissue at a plurality of measurement points of the cross-sectional plane and generating displacement frame data; elasticity information calculation unit for, on the basis of the generated displacement frame data, calculating pieces of elasticity information indicating hardness or softness of the tissue at the plurality of measurement points of the cross-sectional plane and generating elasticity frame data; evaluation image generating unit for generating, as an image for evaluation for evaluating the degree of a disorder of the part to be diagnosed of the object, histograms of at least one of the displacements and the pieces of elasticity information of the tissue at the plurality of measurement points of the cross-sectional plane at different times; and an image display which displays the histograms at the different times.

In this case, the evaluation image generating unit can be adapted to arrange, in time series, a histogram generated for the part to be diagnosed of the object and a histogram previously generated for the same part to be diagnosed of the object and stored in a memory and display the histograms on the image display.

More specifically, a histogram created for the same part to be diagnosed of the object before treatment of the disorder and stored in the memory or a histogram generated for the same part to be diagnosed of the object at the time of a previous diagnosis and stored in the memory can be used as the histogram previously generated and stored in the memory.

According to the present invention, a tester can grasp changes in shape (waveform) among histograms at different times (e.g., histograms arranged and displayed in time series) and changes in peak position among the histograms by referring to the histograms. For example, assume a case where a diffuse disorder of a liver is evaluated. If the disorder progresses from a normal state, hardened tissues appear locally at scattered positions in a soft tissue. At this time, the shape of a histogram changes from a shape having a steep peak near or at a displacement or a piece of elasticity information corresponding to a soft tissue to a broad shape with scattered displacements or pieces of elasticity information. For example, if the disorder progresses, e.g., from hepatitis to liver cirrhosis, since the proportion of hardened tissues increases, the peak of a histogram shifts from the position of a displacement or a piece of elasticity information corresponding to a soft tissue to the position of a displacement or a piece of elasticity information corresponding to a hardened tissue. Accordingly, the tester can more quantitatively perform disorder evaluation of a part to be diagnosed of an object (e.g., evaluation as to how far the disorder of the part to be diagnosed has progressed from the previous test or how much treatment has been effective) by referring to histograms at different times (e.g., histograms arranged and displayed in time series).

Additionally, a model histogram generated in advance so as to correspond to the degree of the disorder of the part to be diagnosed of the object can be stored in the memory, and the model histogram can be displayed together with the plurality of histograms. Alternatively, a coefficient of correlation with the model histogram can be obtained and displayed for each of the plurality of histograms.

According to these configurations, a tester can grasp, with just one look, the degree of a disorder of a part to be diagnosed of an object by contrasting the shape of a model histogram with the shape of a histogram of the part to be diagnosed of the object or contrasting the peak position of the model histogram with the peak position of the histogram of the part to be diagnosed of the object. For example, assume a case where a plurality of stages are set in advance so as to correspond to the degrees of progression of the disorder of the part to be diagnosed. If a plurality of model histograms corresponding to the respective stages are stored in advance in the memory, the tester can easily perform stage determination of the part to be diagnosed of the object by observing the histogram of the part to be diagnosed of the object while contrasting the histogram with the plurality of model histograms. If a coefficient of correlation of the histogram of the part to be diagnosed of the object with each of the plurality of model histograms is obtained and displayed, the tester can quantitatively perform stage determination according to the magnitudes of the coefficients of correlation without contrastive observation of histograms.

The evaluation image generating unit can be provided with statistical processing unit for calculating a piece of statistical processing data of the at least one of the displacements and the pieces of elasticity information of the tissue at the plurality of measurement points of the cross-sectional plane and can display respective pieces of statistical processing data in association with the plurality of histograms. More specifically, at least one of an average value, a median, a mode value, a maximum value, a minimum value, a variance, a standard deviation, and a quartile can be used as the piece of statistical processing data. If the piece of statistical processing data selected via an input interface is any one of an average value, a median, a mode value, a maximum value, and a minimum value, an image indicating a position corresponding to the selected piece of statistical processing data can be displayed for each of the plurality of histograms. On the other hand, if the piece of statistical processing data selected via the input interface is any one of a variance, a standard deviation, and a quartile, an image indicating a section corresponding to the selected piece of statistical processing data can be displayed for each of the plurality of histograms.

According to these configurations, displaying statistical processing data of a histogram is performed in addition to just displaying the histogram. Accordingly, a tester can more quantitatively evaluate the degree of a disorder of a part to be diagnosed. For example, assume a case where a diffuse disorder of a liver is evaluated. If the rough position of an average value, a median, a mode value, a maximum value, or a minimum value of a histogram when a part to be diagnosed is in a normal state is known, and an average value or the like of the part to be diagnosed of an object is smaller than the rough value (position), the tester can grasp that hardened tissues are scattered in a soft tissue. Additionally, since the tester can quantitatively grasp, as a numeric value, by how much the average value or the like is smaller than the rough value of the average value or the like when the part to be diagnosed is in a normal state, the tester can quantitatively determine the degree of spread of the hardened tissues in the soft tissue. For example, a section indicating a variance, a standard deviation, or a quartile (with a range of, e.g., ±σ or ±2σ) is displayed. If the section is broad, displacements or pieces of elasticity information of a tissue vary widely, and the tester can thus grasp that hardened tissues are scattered in a soft tissue.

### Advantageous Effects of Invention

According to the present invention, an image for evaluation for more quantitatively performing disorder evaluation of a part to be diagnosed of an object can be provided.

### BRIEF DESCRIPTION OF DRAWINGS

FIG. 1 is a block diagram showing the overall configuration of an ultrasonic diagnostic device according to a first embodiment of the present invention.

FIG. 2 is a block diagram showing the details of the configuration of an evaluation image construction unit and its surroundings of the ultrasonic diagnostic device according to the first embodiment.

FIG. 3 is a view showing an image display example of the ultrasonic diagnostic device according to the first embodiment.

FIG. 4 is a view showing an image display example of the ultrasonic diagnostic device according to the first embodiment.

FIG. 5 is a view showing an image display example of the ultrasonic diagnostic device according to the first embodiment.

FIG. 6 is a block diagram showing the overall configuration of an ultrasonic diagnostic device according to a second embodiment of the present invention.

FIG. 7 is a block diagram showing the configuration of an evaluation image generating unit of the ultrasonic diagnostic device according to the second embodiment.

FIG. 8 is a view showing an image display example of the ultrasonic diagnostic device according to the second embodiment.

FIG. 9 is a view showing an image display example of the ultrasonic diagnostic device according to the second embodiment.

FIG. 10 is a view showing an image display example of the ultrasonic diagnostic device according to the second embodiment.

FIG. 11 is a view showing an image display example of the ultrasonic diagnostic device according to the second embodiment.

### DESCRIPTION OF EMBODIMENTS

Embodiments of an ultrasonic diagnostic device, a method for generating an image for evaluating a disorder of a part to be diagnosed of an object, and a program for generating an image for evaluating a disorder of a part to be diagnosed of an object, to which the present invention is applied, will be described below. Note that, in the description below, the same functional components are denoted by the same reference numerals and that a redundant description thereof will be omitted.

### First Embodiment

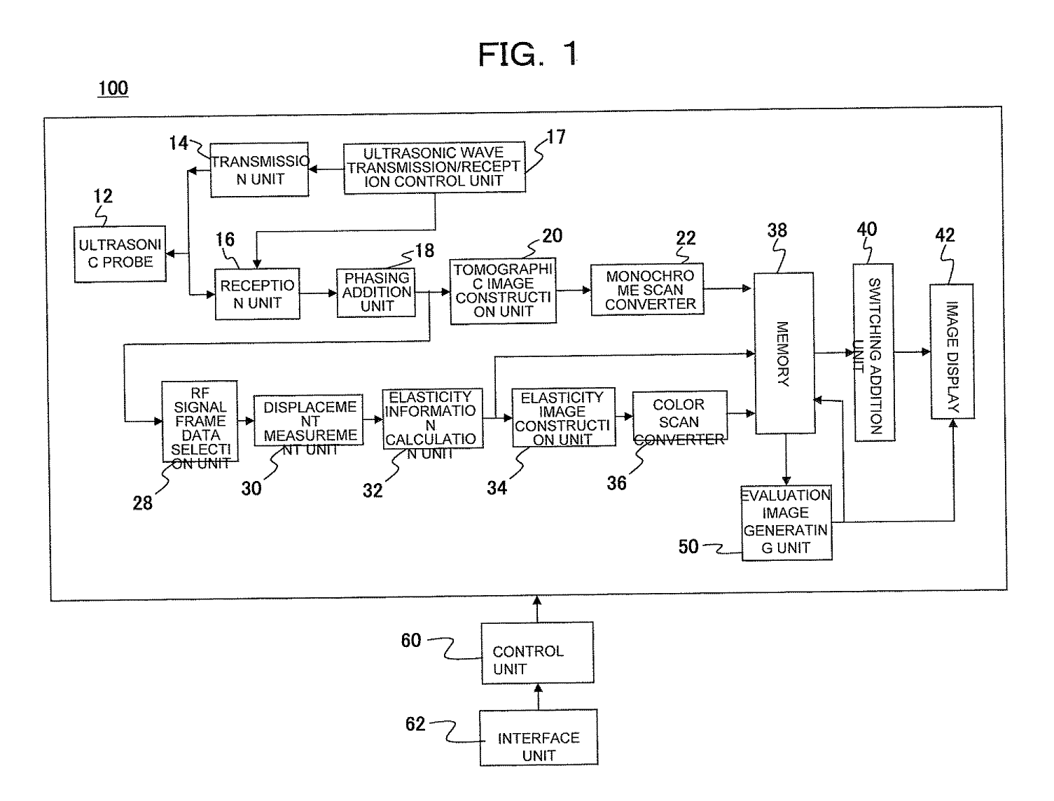

FIG. 1 is a block diagram showing the overall configuration of an ultrasonic diagnostic device according to the first embodiment. The ultrasonic diagnostic device is intended to generate a tomographic image of a tissue at a cross-sectional plane of an object to be examined using ultrasonic waves and obtain elasticity information indicating the hardness or softness of the tissue to generate an elasticity image.

As shown in FIG. 1, an ultrasonic diagnostic device 100 includes an ultrasonic probe 12 which is used in contact with an object, a transmission unit 14 which repeatedly transmits ultrasonic waves to the object at time intervals via the ultrasonic probe 12, a reception unit 16 which receives time-series reflection echo signals originating in the object, an ultrasonic wave transmission/reception control unit 17 which controls the transmission unit 14 and reception unit 16, a phasing addition unit 18 which phases and adds the received reflection echo signals to generate pieces of RF signal frame data in time series, a tomographic image construction unit 20 which performs various signal processes on the pieces of RF signal frame data phased and added by the phasing addition unit 18 to generate a gradation image (e.g., a monochrome tomographic image), and a monochrome scan converter 22 which converts signals output from the tomographic image construction unit 20 so as to be adapted for display on an image display 42.

The ultrasonic diagnostic device 100 also includes an RF signal frame data selection unit 28 which selects a pair of pieces of RF signal frame data acquired at different times from among the pieces of RF signal frame data output from the phasing addition unit 18, a displacement measurement unit 30 which measures a displacement of a tissue at a cross-sectional plane of the object on the basis of the pair of pieces of RF signal frame data to generate displacement frame data, an elasticity information calculation unit 32 which obtains elasticity information (a strain amount or an elastic modulus) indicating the hardness or softness of a living organism tissue of an object in a continuous pressing process on the basis of the displacement frame data measured by the displacement measurement unit 30 to generate elasticity frame data, an elasticity image construction unit 34 which constructs an elasticity image on the basis of the elasticity information calculated by the elasticity information calculation unit 32, and a color scan converter 36 which converts signals output from the elasticity image construction unit 34 so as to be adapted for display on the image display 42.

The ultrasonic diagnostic device 100 also includes a memory 38 which stores tomographic image data output from the monochrome scan converter 22, elasticity image data output from the color scan converter 36, and the like, a switching addition unit 40 which adds or switches between two images on the basis of the tomographic image data and elasticity image data output from the memory 38, the image display 42 that displays an image based on image data output from the switching addition unit 40 and image data for evaluation output from an evaluation image generating unit (to be described later), and an evaluation image generating unit 50 which generates an image for evaluation for evaluating the degree of a disorder of a part to be diagnosed of an object on the basis of elasticity frame data stored in the memory 38. The details of the evaluation image generating unit 50 and other components will be described later.

The ultrasonic diagnostic device 100 also includes a control unit 60 for controlling the above-described components which is composed of, e.g., a CPU (Central Processing Unit) and an interface unit 62, such as a mouse, a keyboard, a touch panel, or a trackball, which gives instructions to control a ROI (Region Of Interest) of an elasticity image, a frame rate, or the like to the control unit 60.

The details of the respective components of the ultrasonic diagnostic device 100 will be described below. The ultrasonic probe 12 is formed such that a large number of transducers are arranged in strip form and is intended to mechanically or electronically perform beam scanning to transmit and receive ultrasonic waves to and from an object. Although not shown, the ultrasonic probe 12 incorporates the transducers that serve as a source of ultrasonic waves and receive reflection echoes. Each transducer is generally formed to have the function of converting input wave transmission signals as pulse waves or continuous waves to ultrasonic waves and emitting the ultrasonic waves and the function of receiving ultrasonic waves emitted from the interior of an object, converting the ultrasonic waves to wave reception signals as electric signals, and outputting the wave reception signals.

The process of, while transmitting/receiving ultrasonic waves by the ultrasonic probe 12, attaching a pressure plate to the ultrasonic probe 12 for effectively providing a stress distribution into a body cavity at a part to be diagnosed of an object such that a surface of the pressure plate fits with an ultrasonic wave transmission/reception surface of the ultrasonic probe 12, bringing a pressing surface constituted by the ultrasonic wave transmission/reception surface of the ultrasonic probe 12 and the pressure plate into contact with the surface of the body of the object, and manually and vertically moving the pressing surface to press the object is generally adopted as the operation of pressing an object in an elasticity image using ultrasonic waves. However, unlike a superficial region such as a mammary gland which is easy to approach from the surface of the body of an object, it may be difficult to press a target tissue by the ultrasonic probe 12 to cause a displacement and a strain amount in an abdominal region such as a liver. For this reason, a displacement and a strain amount caused by heartbeats, arterial pulses, or the like can be used in a case where an abdominal region such as a liver is the target.

The transmission unit 14 is intended to generate a wave transmission pulse for driving the ultrasonic probe 12 and causing the ultrasonic probe 12 to generate ultrasonic waves and set a convergent point of ultrasonic waves transmitted by a wave transmission phasing addition unit incorporated therein to some depth.

The reception unit 16 is intended to amplify reflection echo signals received by the ultrasonic probe 12 with a predetermined gain. Wave reception signals obtained by the amplification and corresponding in number to the respective transducers are input as independent wave reception signals to the phasing addition unit 18. The phasing addition unit 18 is intended to control the phase of a wave reception signal amplified by the reception unit 16 and form an ultrasonic beam for one or more convergent points. The ultrasonic wave transmission/reception control unit 17 is intended to control when to transmit and receive ultrasonic waves.

The tomographic image construction unit 20 subjects RF signal frame from the phasing addition unit 18 to various signal processes, such as gain correction, log correction, wave detection, edge enhancement, and filter processing, and constructs a gradation image (e.g., a monochrome tomographic image) of an object.

The monochrome scan converter 22 is intended to display signals output from the tomographic image construction unit 20 on the image display 42. The monochrome scan converter 22 includes tomographic scanning unit for readout at intervals for the television system and unit for system control, such as an A/D converter which converts signals output from the tomographic image construction unit 20 to digital signals, a plurality of frame memories which store pieces of tomographic image data having undergone digitization in the A/D converter in time series, and a controller which controls the operations of the A/D converter and frame memories.

The RF signal frame data selection unit 28 is responsible for sequentially storing pieces of RF signal frame data chronologically output at a frame rate of the ultrasonic diagnostic device from the phasing addition unit 18 in a frame memory included in the RF signal frame data selection unit 28 (a latest stored piece of RF signal frame data will be referred to as a piece N of RF signal frame data), selecting one piece of RF signal frame data having a different state of pressing (which will be referred to as a piece X of RF signal frame data) from among previous pieces N-1, N-2, N-3, . . . , N-M of RF signal frame data in accordance with a control instruction from the ultrasonic diagnostic device, and outputting one pair of pieces of RF signal frame data, the piece N of RF signal frame data and the piece X of RF signal frame data to the displacement measurement unit 30. Although signals output from the phasing addition unit 18 have been described as a piece of RF signal frame data, for example, the signals may be T and Q signals obtained by subjecting RF signals to complex demodulation.

The displacement measurement unit 30 is intended to perform one-dimensional or two-dimensional correlation processing on the basis of the one pair of RF signal frame data selected by the RF signal frame data selection unit 28, measure a displacement or a motion vector (the direction and magnitude of the displacement) at each measurement point on a tomographic image, and generate displacement frame data. For example, a block matching method or a gradient method is used to detect a motion vector. The block matching method includes dividing an image into blocks of, e.g., N×N pixels, searching in a previous frame for a block most similar to a block of interest in the current frame, and performing prediction coding while referring to the blocks.

The elasticity information calculation unit 32 is intended to calculate a strain amount or an elastic modulus at each measurement point on a tomographic image on the basis of displacement frame data output from the displacement measurement unit 30, generate numeric data of the strain amounts or elastic moduli (elasticity frame data), and output the numeric data to the elasticity image construction unit 34. The calculation of a strain amount performed in the elasticity information calculation unit 32 is computationally performed by spatial differentiation of a displacement. More specifically, letting ΔL be a displacement measured by the displacement measurement unit 30, since a strain amount (S) can be calculated by spatial differentiation of ΔL, the strain amount can be obtained using the equation S=ΔL/ΔX. A Young's modulus Ym, which is an example of an elastic modulus, can be calculated by dividing the stress (pressure) at each calculation point by the strain amount at the calculation point, as given by the following equation: Ymi,j=pressure (stress) i,j/(strain amount i,j) (i,j=1, 2, 3, . . . ).

In the equation, indices i and j represent coordinates of frame data. A pressure applied to the surface of the body of an object can be directly measured by a pressure sensor at a contact surface between the surface of the body of the object and the ultrasonic wave transmission/reception surface of the ultrasonic probe 12. If a displacement and a strain amount are caused in a target tissue by heartbeats, arterial pulses, or the like, the strain amount is used as elasticity information. Note that the elasticity information calculation unit 32 may subject calculated elasticity frame data to various image processes such as smoothing processing in a coordinate plane, contrast optimization processing, and smoothing processing between frames in a direction of time axis and output the processed elasticity frame data as a strain amount.

The elasticity image construction unit 34 includes a frame memory and an image processing unit. The elasticity image construction unit 34 is intended to store pieces of elasticity frame data output in time series from the elasticity information calculation unit 32 in the frame memory and perform image processing on the stored pieces of frame data by the image processing unit.

The color scan converter 36 is composed of a gradation circuit and a hue conversion circuit and includes the hue conversion process of adding hue information of red, green, blue, or the like to each piece of elasticity image frame data output from the elasticity image construction unit 34. Also, the color scan converter 36 may increase the brightness of a region in elasticity image data if a large strain is measured in the region and reduce the brightness of a region in elasticity image data if a small strain is measured in the region, like the monochrome scan converter 22.

The gradation circuit in the color scan converter 36 converts elasticity image frame data output from the elasticity image construction unit 34 so as to have, e.g., a 256-step gradation according to the magnitude of the value of each piece of element data of the elasticity image frame data to generate elasticity gradation frame data. At this time, a region to be subjected to gradation processing is within a region of interest (ROI). However, the region can be arbitrarily changed by a tester via the interface unit 62.

The memory 38 stores tomographic image data output from the monochrome scan converter 22, elasticity frame data output from the elasticity information calculation unit 32, and elasticity image data output from the color scan converter 36. The switching addition unit 40 is unit for receiving monochrome tomographic image data and elasticity image data output from the memory 38 and adding or switching between two images. The switching addition unit 40 is intended to switch between outputting only monochrome tomographic image data or only color elasticity image data and outputting a result of adding and merging the two pieces of image data. As disclosed in, e.g., Japanese Patent Laid-Open No. 2004-135929, which is filed earlier by the applicant of the present application, a color tomographic image may be displayed while being translucently superimposed on a monochrome tomographic image. The monochrome tomographic image here is not limited to a general B-mode image, and a tissue harmonic tomographic image obtained by imaging a harmonic component of a reception signal may be used. Alternatively, a tissue Doppler image may be displayed instead of the monochrome tomographic image.

The image display 42 is composed of a D/A converter which converts image data output from the monochrome scan converter 22 or the color scan converter 36 via the switching addition unit 40 to analog signals and a color TV monitor which receives analog video signals from the D/A converter and displays the analog video signals as an image.

An elasticity image in the ultrasonic diagnostic device 100 is expected to be used not only in diagnosis of a mass lesion such as cancer but also in diagnosis of a diffuse disorder. More specifically, if local hardened tissues such as a tuber are scattered in a surrounding soft tissue in the case of a diffuse disorder, a patchy pattern of the hardened tissues is reflected in an elasticity image. For example, if a disorder progresses, e.g., from hepatitis to liver cirrhosis to cause fibrotic progression, tubers spread into a liver parenchyma, and a patchy pattern of hardened tissues in an elasticity image becomes complicated. A tester observes the elasticity image and makes an evaluation of the degree of the disorder of a part to be diagnosed, the degree of progression of the disorder, effects of treatment of the disorder, and the like on the basis of the state of the patchy pattern of the hardened tissues in the elasticity image.

However, if disorder evaluation is visually performed by a tester, results of the disorder evaluation vary among testers. The process of allowing objective disorder evaluation is thus desired.

The characteristic configuration of the ultrasonic diagnostic device 100 according to the present embodiment made in view of the above will be described below.

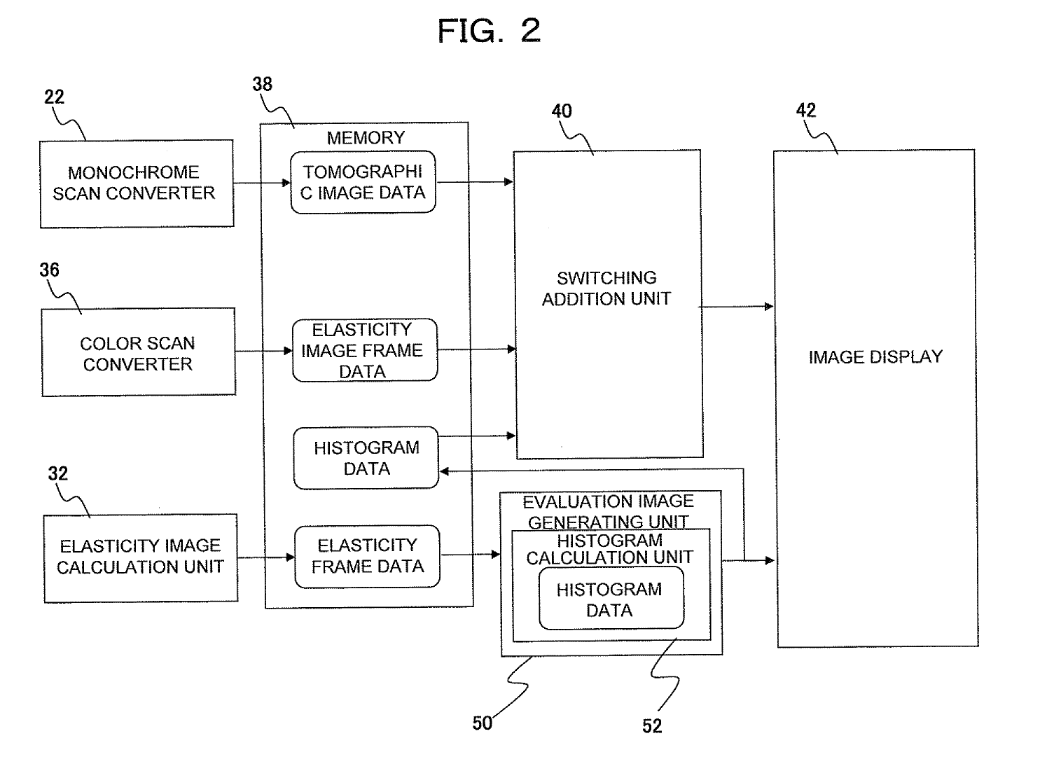

FIG. 2 is a block diagram showing the details of the configuration of the evaluation image generating unit 50 and its surroundings according to the first embodiment. As shown in FIG. 2, the evaluation image generating unit 50 includes a histogram calculation unit 52 which generates a histogram of pieces of elasticity information of a tissue at a plurality of measurement points of an ultrasonic cross-sectional plane of a part to be diagnosed as an image for evaluation for evaluating the degree of a disorder of the part to be diagnosed of an object on the basis of elasticity frame data output from the elasticity information calculation unit 32. The histogram calculation unit 52 can be adapted not only to generate a histogram of pieces of elasticity information of the tissue at the plurality of measurement points but also to generate a histogram of displacements of a tissue at a plurality of measurement points on the basis of displacement frame data output from the displacement measurement unit 30.

The memory 38 can store histogram data generated and output by the histogram calculation unit 52. In the present embodiment, previously generated histograms are stored in the memory 38. More specifically, a previously generated histogram is a histogram generated before treatment of a disorder of the same part to be diagnosed of an object and is stored in the memory or a histogram generated at the time of a previous diagnosis of the same part to be diagnosed of the object and stored in the memory.

The evaluation image generating unit 50 transmits a histogram generated for a part to be diagnosed of an object to the image display 42 to cause the image display 42 to display the histogram and transmits the generated histogram to the memory 38 to cause the memory 38 to store the histogram. The evaluation image generating unit 50 is also adapted to display a histogram generated for a part to be diagnosed of an object and a histogram previously generated for the same part to be diagnosed of the object and stored in the memory 38 as histograms at different times on the image display 42 such that the histograms are arranged in time series. Image display examples by the ultrasonic diagnostic device 100 according to the present embodiment will be described below. Note that display of a histogram will be described below for convenience, an appropriate combination of a tomographic image and an elasticity image can be displayed together with a histogram.

### (First Image Display Example)

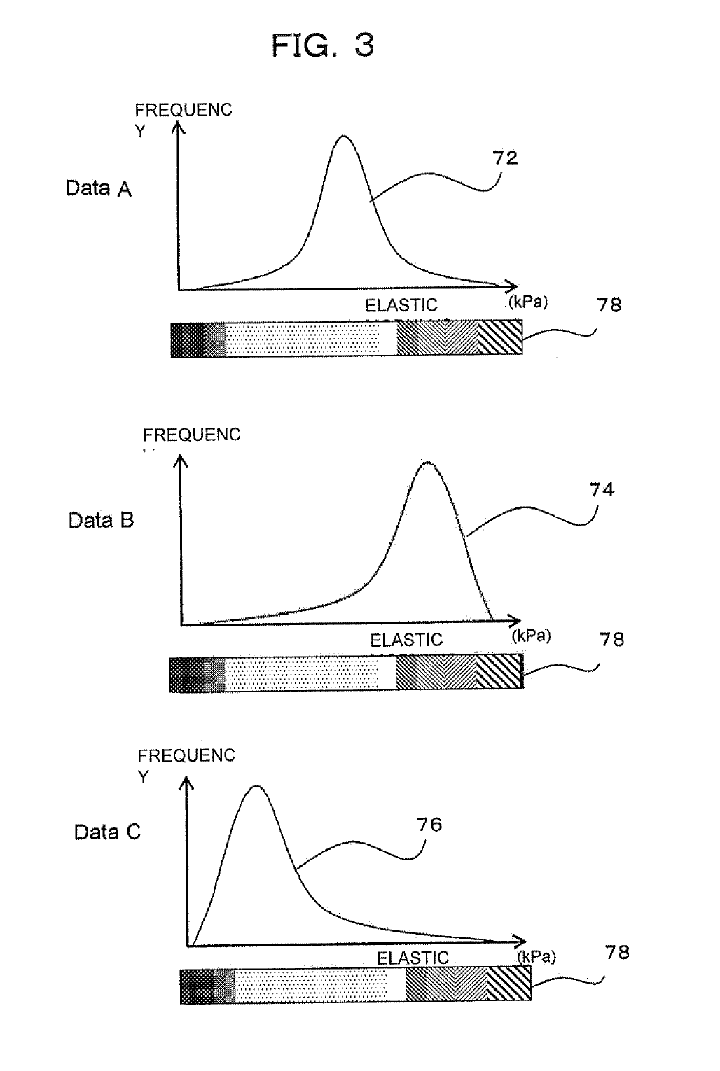

FIG. 3 is a view showing an image display example of the ultrasonic diagnostic device according to the first embodiment. As shown in FIG. 3, on the image display 42, a histogram 72 of data A and a histogram 74 of data B as previous histograms stored in the memory 38 and a histogram 76 of data C as the current histogram are vertically displayed in time series. In the present embodiment, the horizontal axis of each of the histograms 72, 74, and 76 represents elastic moduli expressed in, e.g., 256 gradation steps at a plurality of measurement points of a tissue of an ultrasonic cross-sectional plane while the vertical axis represents the frequencies of the respective elastic moduli. Note that a color map 78 with hues corresponding to the elastic moduli expressed in gradation steps is displayed under each of the histograms 72, 74, and 76.

For example, assume a case where a diffuse disorder of a liver is evaluated. Assume that the histogram 72 of the data A is a histogram generated for a part to be diagnosed of an object at a diagnosis time (diagnosis time a) and stored in the memory 38 and that the histogram 74 of the data B is a histogram generated at a diagnosis time (diagnosis time b) after a lapse of several months (e.g., six months) or about one year from the diagnosis time a and stored in the memory 38.

A tester compares the histogram 72 of the data A and the histogram 74 of the data B. The tester can grasp, from the peak of the waveform shifted to the right, i.e., in a direction toward higher elastic moduli, that local hardened tissues were scattered in a soft tissue during a period from the diagnosis time a to the diagnosis time b. With this comparison, the tester can quantitatively determine how far the disorder of the part to be diagnosed had progressed from the previous test.

Assume here that the histogram 76 of the data C is a histogram which is currently generated with the disorder treated after the diagnosis time b. The tester compares the histogram 74 of the data B and the histogram 76 of the data C. The tester can grasp, from the peak of the waveform shifted to the left, i.e., in a direction toward lower elastic moduli, that scattered hardened tissues have decreased and that the proportion of a soft tissue has increased. As a result, the tester can quantitatively determine that the treatment after the diagnosis time b has produced a predetermined effect.

### (Second Image Display Example)

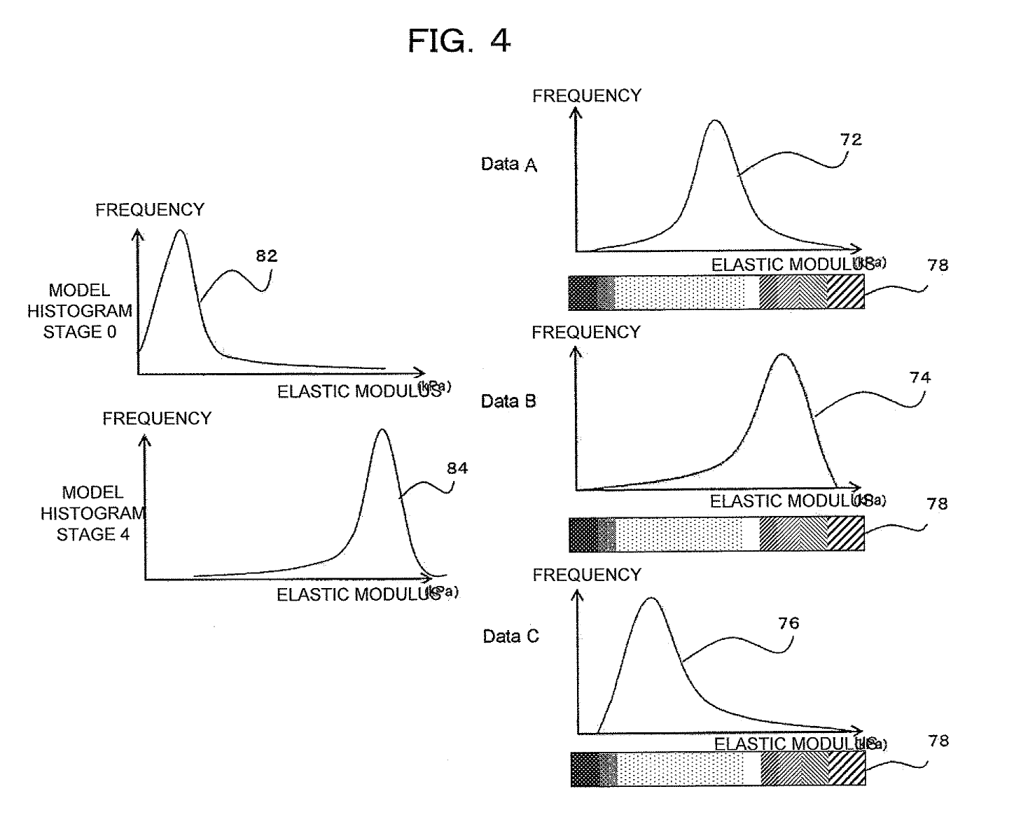

FIG. 4 is a view showing an image display example of the ultrasonic diagnostic device according to the first embodiment. In this example, model histograms which are generated so as to correspond to the degrees of the disorder of the part to be diagnosed of the object are stored in advance in the memory 38, and the model histograms are displayed together with the histograms 72, 74, and 76.

For example, assume a case where a plurality of stages (stages 0 to 4) are set in advance so as to correspond to the degrees of progression of the disorder of the part to be diagnosed of the object. A plurality of histograms corresponding to the respective stages are stored as model histograms in advance in the memory. As shown in FIG. 4, a model histogram 82 for stage 0 and a model histogram 84 for stage 4 are displayed together with the histograms 72, 74, and 76.

According to this example, the tester can easily perform stage determination of the part to be diagnosed of the object by observing the histograms 72, 74, and 76 while contrasting the histograms 72, 74, and 76 with the model histograms 82 and 84. For example, the tester can grasp, by contrasting the histogram 74 of the diagnosis time b with the model histograms 82 and 84, that the part to be diagnosed at the diagnosis time b had not reached stage 4 but that the disorder was progressing. The tester can also grasp, by contrasting the histogram 76 of the diagnosis time c with the model histograms 82 and 84, that the part to be diagnosed at the diagnosis time c is close to stage 0 and that the treatment of the disorder has been effective. Note that although only the model histogram for stage 0 and the model histogram for stage 4 are displayed in this example, the model histograms for the other stages can also be displayed.

### (Third Image Display Example)

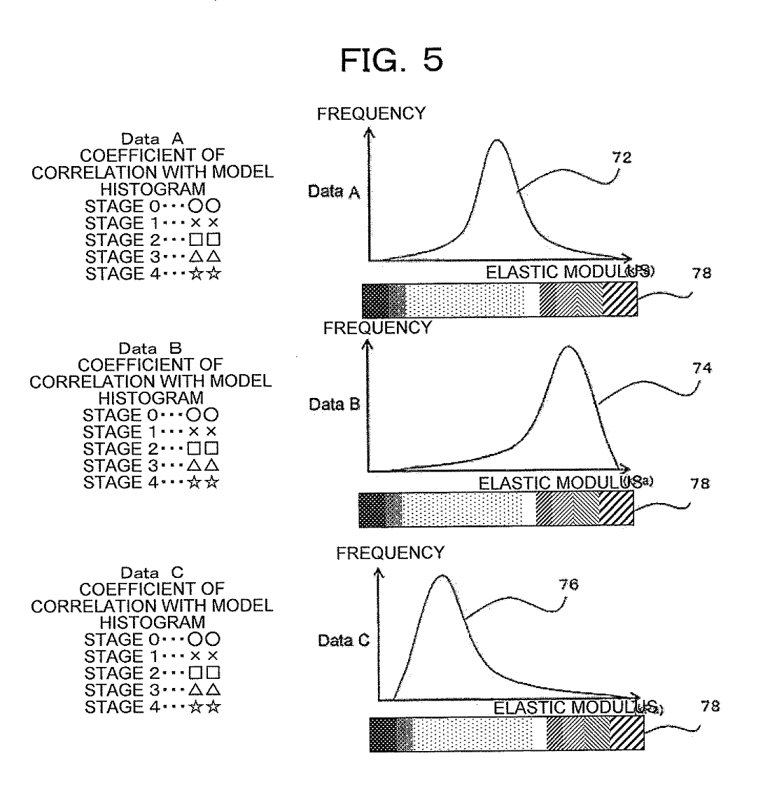

FIG. 5 is a view showing an image display example of the ultrasonic diagnostic device according to the first embodiment. In this example, model histograms which are generated so as to correspond to the degrees of the disorder of the part to be diagnosed of the object are stored in advance in the memory 38, and respective coefficients of correlation with the model histograms are obtained for each of the histograms 72, 74, and 76 and are displayed.

For example, assume a case where a plurality of stages (stages 0 to 4) are set in advance so as to correspond to the degrees of progression of the disorder of the part to be diagnosed. A plurality of histograms corresponding to the respective stages are stored as model histograms in advance in the memory. As shown in FIG. 5, coefficients of correlation with the model histograms for the respective stages are obtained for each of the histograms 72, 74, and 76 and are displayed.

According to this example, the tester can grasp that each of the histograms 72, 74, and 76 has a strong probability of corresponding to a stage with a highest coefficient of correlation with the histogram and can perform quantitative stage determination.

For example, a coefficient of correlation or the like can be displayed as a coincidence rate indicating how much the current histogram 76 coincides with the previous histogram 72 or 74. Although the histograms are vertically arranged in FIGS. 3 to 5, the histograms may be horizontally arranged or the tester may rearrange the histograms. The tester can switch between displaying and hiding each histogram. Even if a plurality of regions of interest (ROIs) are set on the same frame, distributions of elasticity information for different regions on the same cross-section can be easily compared by arranging and displaying a plurality of histograms corresponding to the respective ROIs.

Note that although the above-described embodiment has primarily described an ultrasonic diagnostic device and a method for generating an image for evaluating a disorder of a part to be diagnosed of an object, the present invention is not limited to this. The present invention can be implemented as a program for generating an image for evaluating a disorder of a part to be diagnosed of an object which can be installed in, e.g., an ultrasonic diagnostic device or a computer such as a PC and can be executed.

The program for generating an image for evaluating a disorder of a part to be diagnosed of an object includes a step of generating, as an image for evaluation for evaluating the degree of the disorder of the part to be diagnosed of the object, histograms of at least one of displacements of a tissue at a plurality of measurement points of a cross-sectional plane generated on the basis of RF signal frame data at the cross-sectional plane of the part to be diagnosed of the object which is obtained from reflection echo signals measured by an ultrasonic probe which transmits/receives ultrasonic waves to/from the object and pieces of elasticity information indicating the hardness or softness of the tissue at the plurality of measurement points of the cross-sectional plane which are generated on the basis of the displacements of the tissue at the plurality of measurement points at different times and a step of displaying the histograms at the different times on an image display. The step of displaying the histograms can be adapted to include arranging a histogram generated for the part to be diagnosed of the object and a histogram previously generated for the same part to be diagnosed of the object and stored in a memory in time series and displaying the histograms on the image display.

In this case, like the ultrasonic diagnostic device described above, a tester can grasp changes in shape (waveform) among the histograms and changes in peak position among the histograms by referring to the histograms arranged and displayed in time series. For example, assume a case where a diffuse disorder of a liver is evaluated. If the disorder progresses from a normal state, hardened tissues appear locally at scattered positions in a soft tissue. At this time, the shape of a histogram changes from a shape having a steep peak near or at a displacement or a piece of elasticity information corresponding to a soft tissue to a broad shape with scattered displacements or pieces of elasticity information. For example, if the disorder progresses, e.g., from hepatitis to liver cirrhosis, since the proportion of hardened tissues increases, the peak of a histogram shifts from the position of a displacement or a piece of elasticity information corresponding to a soft tissue to the position of a displacement or a piece of elasticity information corresponding to a hardened tissue. Accordingly, the tester can more quantitatively perform disorder evaluation of a part to be diagnosed of an object (e.g., evaluation as to how far the disorder of the part to be diagnosed has progressed from the previous test or how much treatment has been effective) by referring to histograms arranged and displayed in time series.

### Second Embodiment

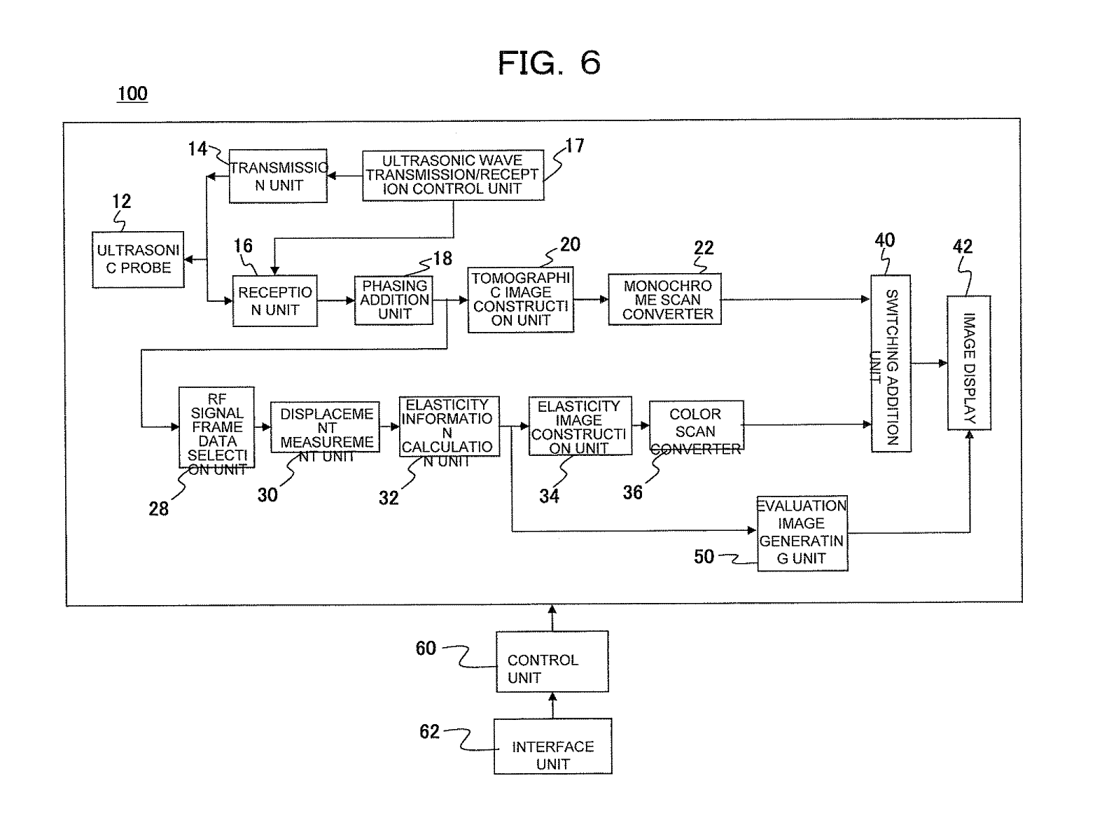

FIG. 6 is a block diagram showing the overall configuration of an ultrasonic diagnostic device according to a second embodiment of the present invention. The second embodiment is different from the first embodiment in that the memory 38 is not provided and that an evaluation image generating unit 50 includes a statistical processing unit. Since the second embodiment is the same as the first embodiment in the other points, a redundant description thereof will be omitted. Note that although the second embodiment will be described in the context of a case without the memory 38, the memory 38 may be provided.

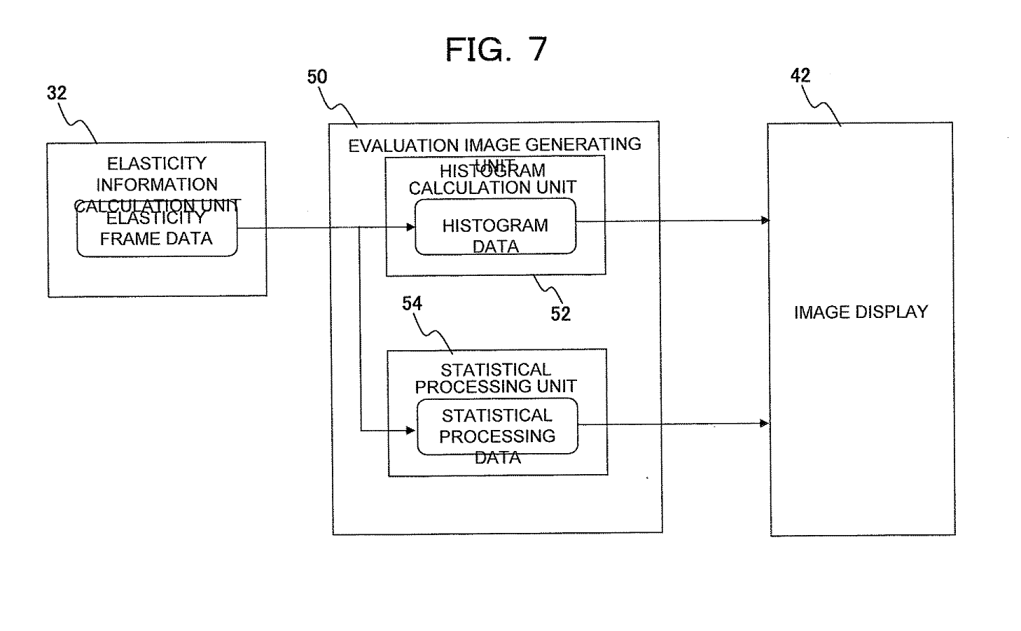

FIG. 7 is a diagram showing the configuration of the evaluation image generating unit 50 according to the present embodiment. As shown in FIG. 7, the evaluation image generating unit 50 includes a histogram calculation unit 52 which generates a histogram of pieces of elasticity information of a tissue at a plurality of measurement points of an ultrasonic cross-sectional plane of a part to be diagnosed of an object as an image for evaluation for evaluating the degree of a disorder of the part to be diagnosed on the basis of elasticity frame data output from an elasticity information calculation unit 32 and a statistical processing unit 54 which calculates statistical processing data of the pieces of elasticity information of the tissue at the plurality of measurement points of the ultrasonic cross-sectional plane. The statistical processing unit 54 can be adapted not only to calculate the statistical processing data of the pieces of elasticity information of the tissue at the plurality of measurement points of the ultrasonic cross-sectional plane but also to calculate statistical processing data of displacements of the tissue at the plurality of measurement points on the basis of displacement frame data output from a displacement measurement unit 30.

The statistical processing unit 54 calculates, as statistical processing data, at least one of an average value, a median, a mode value, a maximum value, a minimum value, a variance, a standard deviation, and a quartile of pieces of elasticity information of a tissue at a plurality of measurement points. The evaluation image generating unit 50 displays statistical processing data generated by the statistical processing unit 54 in association with a histogram generated by the histogram calculation unit 52. Image display examples by an ultrasonic diagnostic device 100 according to the present embodiment will be described below.

### (Fourth Image Display Example)

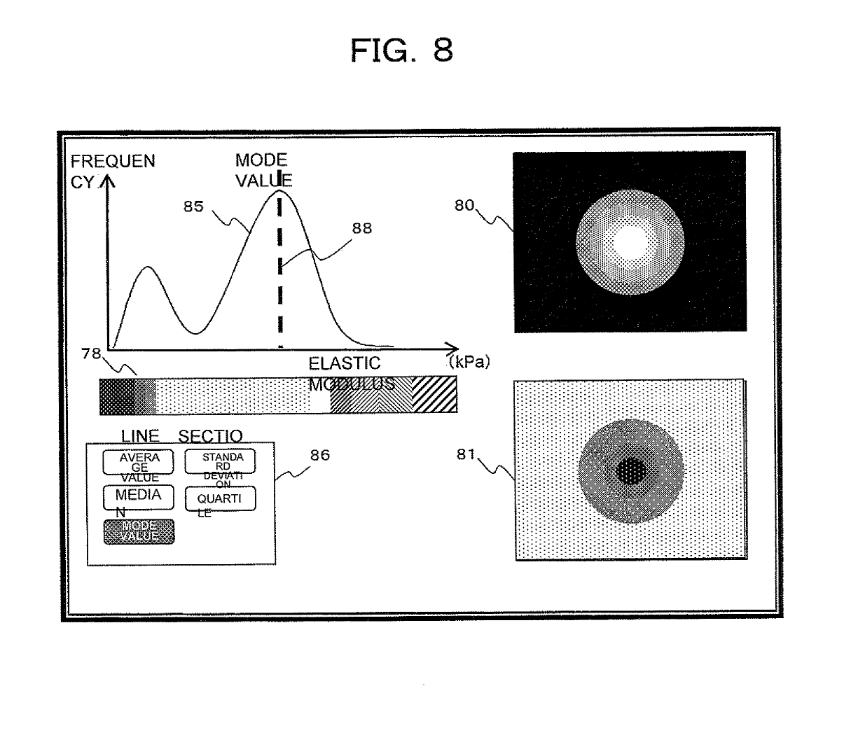

FIG. 8 is a view showing an image display example of the ultrasonic diagnostic device according to the second embodiment. As shown in FIG. 8, a B-mode image 80 as a tomographic image, an elasticity image 81, and a histogram 85 which is generated by the histogram calculation unit 52 are displayed on an image display 42. In the present embodiment, the horizontal axis of the histogram 85 represents elastic moduli expressed in, e.g., 256 gradation steps at a plurality of measurement points of a tissue of an ultrasonic cross-sectional plane while the vertical axis represents the frequencies of the respective elastic moduli. Note that a color map 78 with hues corresponding to the elastic moduli expressed in gradation steps is displayed under the histogram 85.

Select buttons 86 for an average value, a median, a mode value, a standard deviation, and a quartile as respective pieces of statistical processing data are displayed on the image display 42. The select buttons 86 can be selected via an interface unit 62. FIG. 8 shows a display example when a tester has selected a mode value via the interface unit 62. In this case, a line image 88 indicating a position corresponding to the selected mode value is displayed on the histogram 85. The line image 88 is vertically drawn at a position corresponding to the mode value of the horizontal axis (elastic modulus) of the histogram in parallel with the vertical axis of the histogram.

If a piece of statistical processing data selected via the interface unit 62 is one of an average value, a median, a mode value, a maximum value, and a minimum value, an image indicating a position corresponding to the selected piece of statistical processing data is similarly displayed on the histogram.

According to this example, displaying statistical processing data of a histogram is performed in addition to just displaying the histogram. Accordingly, a tester can more quantitatively evaluate the degree of a disorder of a part to be diagnosed. For example, assume a case where a diffuse disorder of a liver is evaluated. If the rough position of an average value, a median, a mode value, a maximum value, or a minimum value of a histogram when a part to be diagnosed is in a normal state is known, and an average value or the like of the part to be diagnosed of an object is smaller than the rough value (position), the tester can grasp that hardened tissues are scattered in a soft tissue. Additionally, since the tester can quantitatively grasp, as a numeric value, by how much the average value or the like is smaller than the rough value of the average value or the like when the part to be diagnosed is in a normal state, the tester can quantitatively determine the degree of spread of the hardened tissues in the soft tissue.

### (Fifth Image Display Example)

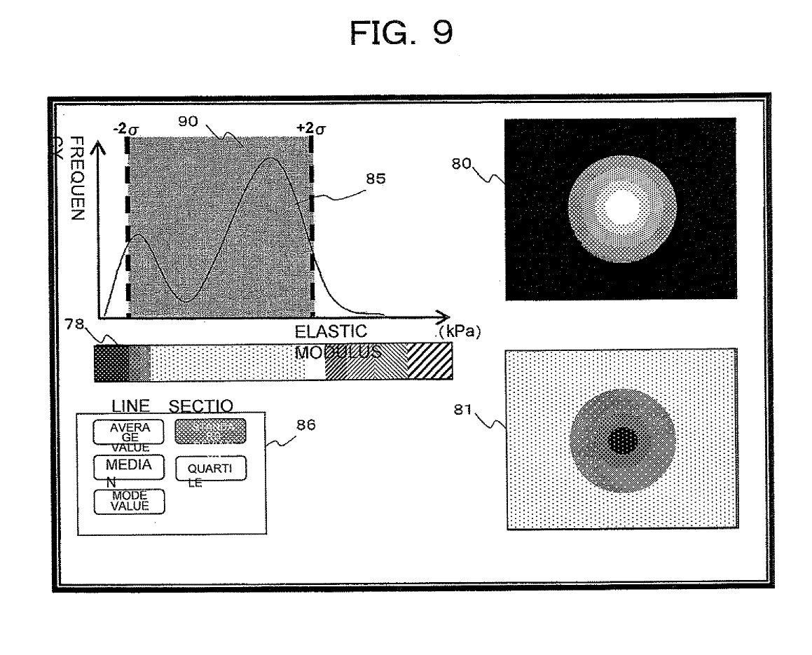

FIG. 9 is a view showing an image display example of the ultrasonic diagnostic device according to the second embodiment. The display example shows a display example when a tester has selected a standard deviation via the interface unit 62. In this case, a section image 90 indicating a section corresponding to the selected standard deviation is displayed on the histogram 85. The section image 90 is drawn with a hue in a section with a range of ±2σ of the histogram 85. An arbitrary section (with a range of, e.g., ±σ) can be selected via the interface unit 62 as the section by the tester.

If a piece of statistical processing data selected via the interface unit 62 is one of a variance, a standard deviation, and a quartile, an image indicating a section corresponding to the selected piece of statistical processing data is similarly displayed on the histogram.

As described above, a section indicating a variance, a standard deviation, or a quartile (with a range of, e.g., ±σ or ±2σ) is displayed. If the section is broad, displacements or pieces of elasticity information of a tissue vary widely, and it can thus be grasped that hardened tissues are scattered in a soft tissue. Also, since how wide a histogram extends with respect to a rough section indicating a standard deviation or the like when a part to be diagnosed is in a normal state can be quantitatively grasped as a numeric value, the degree of spread of hardened tissues in a soft tissue can be quantitatively grasped.

### (Sixth Image Display Example)

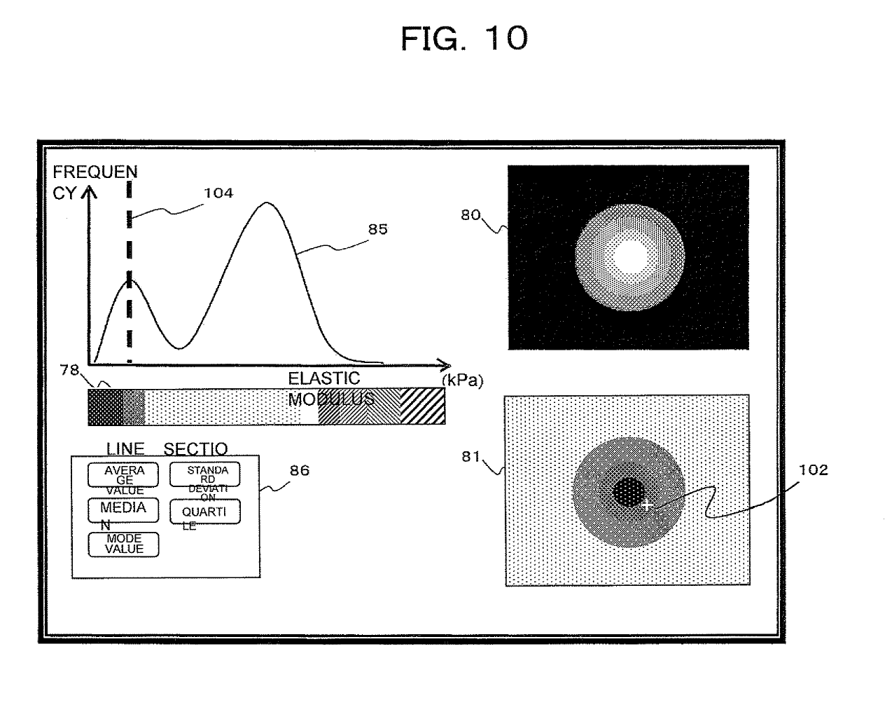

FIG. 10 is a view showing an image display example of the ultrasonic diagnostic device according to the second embodiment. In the display example, the evaluation image generating unit displays a position corresponding to a displacement or a piece of elasticity information at a point selected on the elasticity image 81 via the interface unit 62 as a line image on the histogram 85.

As shown in FIG. 10, when a tester selects a point on the elasticity image 81 with a cursor 102 which can be moved via the interface unit 62, a line image 104 is displayed on the histogram 85 so as to correspond to an elastic modulus of a tissue at the selected point. The line image 104 is vertically drawn at a position corresponding to the selected point of the horizontal axis (elastic modulus) of the histogram in parallel with the vertical axis of the histogram.

According to this example, since the tester can easily grasp to which position of the histogram the tissue of interest on the elasticity image being referred to corresponds, association of the elasticity image with the histogram is easy. Although only one point to be selected is set in this example, a plurality of points to be selected can be set.

### (Seventh Image Display Example)

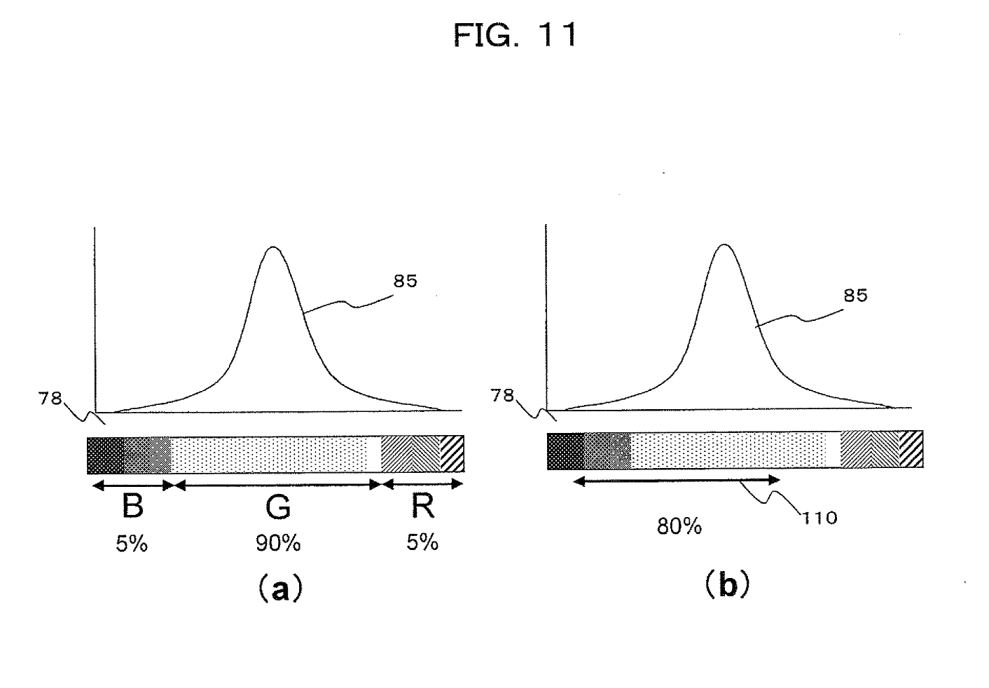

FIG. 11 are views showing image display examples of the ultrasonic diagnostic device according to the second embodiment. In each display example, a color map with hues corresponding to the respective magnitudes of displacements or pieces of elasticity information in a histogram is displayed together with the histogram, and the evaluation image generating unit displays the proportion of the frequency of displacements or pieces of elasticity information within a preset range or a range set via the interface unit 62 of the color map to the total frequency.

FIG. 11(a) shows the proportion of the frequency of pieces of elastic modulus within each of preset ranges of the color map 78 to the total frequency. The color map 78 has blue (B), green (G), and red (R) hues in the order of elastic modulus from smallest to largest, and the preset ranges are respective ranges for blue (B), green (G), and red (R). This example shows that the frequency of elastic moduli included in blue (B) is 5%, that the frequency of elastic moduli included in green (G) is 90%, and that the frequency of elastic moduli included in red (R) is 5%.

In contrast, FIG. 11(b) shows the proportion of the frequency of elastic moduli within a range set via the interface unit 62 of the color map 78 to the total frequency. FIG. 11(b) shows that the proportion of the frequency of elastic moduli within a range 110 of the color map 78 which is set via the interface unit 62 by a tester to the total frequency is 80%.

According to these examples, a tester can recognize, as a numeric value, the proportion of the frequency of elastic moduli within a preset range or an arbitrary range of a color map and can use the proportion as an index to disorder evaluation. More quantitative disorder evaluation of a part to be diagnosed of an object, such as determining that a large number of hardened tissues are included in a part to be diagnosed and that the part to be diagnosed has a strong probability of having a disorder, for example, if the frequency of elastic moduli within the range for blue (B) in FIG. 11(a) is higher than a given threshold value, can be performed.

Note that, in the second embodiment, a model histogram can be displayed together with the histogram 85, in the same manner as described with reference to the second image display example of the first embodiment. In this case, a tester can easily perform stage determination for a part to be diagnosed of an object by observing the histogram 85 while contrasting the histogram 85 with the model histogram.

In the second embodiment, a coefficient of correlation of the histogram 85 with a model histogram can be obtained and displayed, in the same manner as described with reference to the third image display example of the first embodiment. In this case, a tester can grasp that each of the histograms 72, 74, and 76 has a strong probability of corresponding to a stage with a highest coefficient of correlation with the histogram and can perform quantitative stage determination.

In the first embodiment, a piece of statistical processing data may also be displayed in association with each of a plurality of displayed histograms, in the same manner as described with reference to the third image display example of the second embodiment.

In the first embodiment, an elasticity image generated for a part to be diagnosed of an object and an elasticity image previously generated for the same part to be diagnosed of the object and stored in a memory can be displayed in association with each of a plurality of displayed histograms, and an image indicating a position corresponding to a displacement or a piece of elasticity information at a point selected on the displayed elasticity images via an input interface can be displayed on the corresponding histogram, in the same manner as described with reference to the fourth image display example of the second embodiment.

In the first embodiment, a color map can be displayed in association with each of a plurality of displayed histograms, and the proportion of the frequency of displacements or pieces of elasticity information within a preset range or a range set via an interface unit of each color map to the total frequency can be displayed, in the same manner as described with reference to the fifth image display example of the second embodiment.

### REFERENCE SIGNS LIST

12 ultrasonic probe, 18 phasing addition unit, 20 tomographic image construction unit, 28 RF signal frame data selection unit, 30 displacement measurement unit, 32 elasticity information calculation unit, 34 elasticity image construction unit, 38 memory, 42 image display, 50 evaluation image generating unit, 54 statistical processing unit, 62 interface unit, 72, 74, 76, 85 histogram, 78 color map, 82, 84 model histogram, 81 elasticity image, 100 ultrasonic diagnostic device

1. An ultrasonic diagnostic device, comprising:
an ultrasonic probe for transmitting/receiving ultrasonic waves to/from an object;
reception processing unit for receiving a reflection echo signal measured by the ultrasonic probe and generating RF signal frame data at a cross-sectional plane of a part to be diagnosed of the object;
displacement measurement unit for, on the basis of one pair of pieces of RF signal frame data having different states of pressing against a tissue at the cross-sectional plane, measuring displacements of the tissue at a plurality of measurement points of the cross-sectional plane and generating displacement frame data;
elasticity information calculation unit for, on the basis of the generated displacement frame data, calculating pieces of elasticity information indicating hardness or softness of the tissue at the plurality of measurement points of the cross-sectional plane and generating elasticity frame data;
evaluation image generating unit for generating, as an image for evaluation for evaluating the degree of a disorder of the part to be diagnosed of the object, histograms of at least one of the displacements and the pieces of elasticity information of the tissue at the plurality of measurement points of the cross-sectional plane at different times; and
an image display which displays the histograms at the different times.

2. The ultrasonic diagnostic device according to claim 1, wherein
the evaluation image generating unit arranges, in time series, a histogram generated for the part to be diagnosed of the object and a histogram previously generated for the same part to be diagnosed of the object and stored in a memory and displays the histograms on the image display.

3. The ultrasonic diagnostic device according to claim 2, wherein
the histogram previously generated and stored in the memory is a histogram created for the same part to be diagnosed of the object before treatment of the disorder and stored in the memory or a histogram generated for the same part to be diagnosed of the object at the time of a previous diagnosis and stored in the memory.

4. The ultrasonic diagnostic device according to claim 3, wherein
the memory stores a model histogram generated in advance so as to correspond to the degree of the disorder of the part to be diagnosed of the object, and
the model histogram is displayed together with the plurality of histograms.

5. The ultrasonic diagnostic device according to claim 3, wherein
the memory stores a model histogram generated in advance so as to correspond to the degree of the disorder of the part to be diagnosed of the object, and
a coefficient of correlation with the model histogram is obtained and displayed for each of the plurality of histograms.

6. The ultrasonic diagnostic device according to claim 1, wherein
the evaluation image generating unit includes statistical processing unit for calculating a piece of statistical processing data of the at least one of the displacements and the pieces of elasticity information of the tissue at the plurality of measurement points of the cross-sectional plane and displays respective pieces of statistical processing data in association with the plurality of histograms.

7. The ultrasonic diagnostic device according to claim 6, wherein
the piece of statistical processing data is at least one of an average value, a median, a mode value, a maximum value, a minimum value, a variance, a standard deviation, and a quartile,
if a piece of statistical processing data selected via an input interface is any one of an average value, a median, a mode value, a maximum value, and a minimum value, an image indicating a position corresponding to the selected piece of statistical processing data is displayed for each of the plurality of histograms, and
if the piece of statistical processing data selected via the input interface is any one of a variance, a standard deviation, and a quartile, an image indicating a section corresponding to the selected piece of statistical processing data is displayed for each of the plurality of histograms.

8. The ultrasonic diagnostic device according to claim 2, comprising:
elasticity image generation unit for generating an elasticity image on the basis of the elasticity frame data and displaying the elasticity image on the image display, wherein
the evaluation image generating unit displays an elasticity image generated for the part to be diagnosed of the object and an elasticity image previously generated for the same part to be diagnosed of the object and stored in the memory in association with each of the plurality of histograms and displays an image indicating a position corresponding to a displacement or a piece of elasticity information at a point selected on the displayed elasticity images via the input interface on the corresponding histogram.

9. The ultrasonic diagnostic device according to claim 1, wherein
a color map with hues corresponding to respective magnitudes of displacements or pieces of elasticity information of each of the plurality of histograms is displayed together with the histogram, and
the evaluation image generating unit displays a proportion of a frequency of displacements or pieces of elasticity information within a preset range or a range set via the input interface of each of the color maps to a total frequency.

10. An ultrasonic diagnostic device, comprising:
an ultrasonic probe which transmits/receives ultrasonic waves to/from an object;
reception processing unit for receiving a reflection echo signal measured by the ultrasonic probe and generating RF signal frame data at a cross-sectional plane of a part to be diagnosed of the object;
displacement measurement unit for, on the basis of one pair of pieces of RF signal frame data having different states of pressing against a tissue at the cross-sectional plane, measuring displacements of the tissue at a plurality of measurement points of the cross-sectional plane and generating displacement frame data;
elasticity information calculation unit for, on the basis of the generated displacement frame data, calculating pieces of elasticity information indicating hardness or softness of the tissue at the plurality of measurement points of the cross-sectional plane and generating elasticity frame data;
evaluation image generating unit for generating, as an image for evaluation for evaluating the degree of a disorder of the part to be diagnosed of the object, a histogram of at least one of the displacements and the pieces of elasticity information of the tissue at the plurality of measurement points of the cross-sectional plane; and
an image display which displays the histogram, wherein
the evaluation image generating unit includes statistical processing unit for calculating a piece of statistical processing data of the at least one of the displacements and the pieces of elasticity information of the tissue at the plurality of measurement points of the cross-sectional plane and displays the piece of statistical processing data in association with the histogram.

11. The ultrasonic diagnostic device according to claim 10, wherein
the piece of statistical processing data is at least one of an average value, a median, a mode value, a maximum value, a minimum value, a variance, a standard deviation, and a quartile,
if a piece of statistical processing data selected via an input interface is any one of an average value, a median, a mode value, a maximum value, and a minimum value, an image indicating a position corresponding to the selected piece of statistical processing data is displayed for the histogram, and
if the piece of statistical processing data selected via the input interface is any one of a variance, a standard deviation, and a quartile, an image indicating a section corresponding to the selected piece of statistical processing data is displayed for the histogram.

12. A method for generating an image for evaluating a disorder of a part to be diagnosed of an object, comprising:
receiving a reflection echo signal measured by an ultrasonic probe which transmits/receives ultrasonic waves to/from the object and generating RF signal frame data at a cross-sectional plane of the part to be diagnosed of the object;
measuring displacements of the tissue at a plurality of measurement points of the cross-sectional plane and generating displacement frame data, on the basis of one pair of pieces of RF signal frame data having different states of pressing against a tissue at the cross-sectional plane;
calculating pieces of elasticity information indicating hardness or softness of the tissue at the plurality of measurement points of the cross-sectional plane and generating elasticity frame data, on the basis of the generated displacement frame data;
generating, as an image for evaluation for evaluating the degree of the disorder of the part to be diagnosed of the object, histograms of at least one of the displacements and the pieces of elasticity information of the tissue at the plurality of measurement points of the cross-sectional plane at different times; and
displaying the histograms at the different times on an image display.

13. The method for generating the image for evaluating the disorder of the part to be diagnosed of the object, according to claim 12, wherein
displaying the histograms comprises arranging, in time series, a histogram generated for the part to be diagnosed of the object and a histogram previously generated for the same part to be diagnosed of the object and stored in a memory and displaying the histograms on the image display.

14. (canceled)

15. (canceled)
